(19) United States
(12) Patent Application Publication
(10) Pub. No.: US 2018/0303440 A1
(43) Pub. Date: Oct. 25, 2018

(54) AUTOMATICALLY CONTROLLED X-RAY IRRADIATION FIELD LIMITING DEVICE

(71) Applicant: DIGIT MEDICAL CORPORATION, Osan-si (KR)

(72) Inventor: Chang Sik SUNG, Yongin-si (KR)

(21) Appl. No.: 15/957,226

(22) Filed: Apr. 19, 2018

(30) Foreign Application Priority Data
Apr. 19, 2017 (KR) 10-2017-0050331
Apr. 4, 2018 (KR) 10-2018-0039410

(51) Int. Cl. A61B 6/06; A61B 6/00

(52) U.S. Cl. CPC A61B 6/06; A61B 6/542; A61B 6/4035; A61B 6/563; A61B 6/547

(57) ABSTRACT

An automatically controlled X-ray irradiation field limiting device includes a main body disposed in front of an X-ray generator for generating X-rays, an X-ray irradiation field limiting unit mounted to the main body and configured to adjust an X-ray irradiation field formed by the X-rays generated from the X-ray generator in accordance with an opening area of a variable window, a drive unit configured to operate the X-ray irradiation field limiting unit to adjust the X-ray irradiation field, a light irradiation field device mounted inside the main body and configured to form a light irradiation field, a distance information acquiring device mounted on the main body and configured to measure and acquire a distance between a focus of the X-ray generator and the subject, and a controller configured to control an operation of the drive unit based on the distance received from the distance information acquiring device.

### TECHNICAL FIELD

The present invention relates to an X-ray irradiation field limiting device, and more particularly, to an automatically controlled X-ray irradiation field limiting device capable of automatically adjusting and maintaining an X-ray irradiation field at a preset X-ray irradiation field size depending on the distance between a focus of an X-ray generator and a subject.

### BACKGROUND ART

In an X-ray apparatus inspection for a region where an important organ of a human body is present, an unnecessarily large X-ray irradiation field may increase the damage of the important organ due to the radiation exposure. Thus, an X-ray apparatus includes various safety devices for reducing the exposure dose to a subject and an operator. An example of the safety devices includes an X-ray irradiation field limiting device. The X-ray irradiation field limiting device is a basic component of an X-ray apparatus and is a variable diaphragm disposed in front of an X-ray generator or an X-ray tube to make sure that an X-ray is irradiated only on a limited irradiation range. The X-ray irradiation field limiting device is also referred to as a collimator.

Korean Patent No. 10-1362464 entitled “X-rays field controlling device having dose-area product meter embedded therein (PCT International Application Publication No. WO 2013/109081 A1) discloses an X-ray irradiation field limiting device disposed in front of an X-ray generator to control an X-ray irradiation field. The X-ray irradiation field limiting device includes a pair of first X-ray limiting blades arranged parallel to each other along a first direction, and a pair of second X-ray limiting blades arranged in parallel to each other along a second direction orthogonal to the first direction. Each of the first and second X-ray limiting blades is moved toward or away from each other to remove X-rays generated outside the focus of an X-ray generator and to make sure that only the X-rays generated at the focus are irradiated to a desired region of a subject. Accordingly, the amount of X-rays irradiated to an unnecessary region of a subject is reduced, thereby reducing the exposure dose and improving the contrast of an image and the image quality. The contents disclosed in the above patent document are incorporated herein by reference.

The X-ray irradiation field limiting device as described above changes the X-ray irradiation field size according to the distance between the focus of the X-ray generator and the subject. However, it is difficult for the X-ray irradiation field limiting device to maintain a preset X-ray irradiation field size and to adjust an exposure dose by moving the first and second X-ray limiting blades in accordance with the distance between the focus and the subject. In addition, it is necessary to measure an area dose and a standard dose at the same time in order to manage the quality of the X-ray generator and the exposure dose. Recently, the risk of a cumulative radiation dose or an accumulated dose is increasing due to the increase of radiation examination in the medical field. Therefore, a demand has existed for an automatically controlled X-ray irradiation field limiting device capable of automatically adjusting an X-ray irradiation field depending on the distance between an X-ray generator and a subject, thereby maintaining a preset X-ray irradiation field size and reducing a cumulative radiation dose.

### SUMMARY

The present invention provides a novel automatically controlled X-ray irradiation field limiting device capable of meeting the above demand.

According to one embodiment of the present invention, there is provided an automatically controlled X-ray irradiation field limiting device, including: a main body disposed in front of an X-ray generator for generating X-rays; an X-ray irradiation field limiting unit mounted to the main body and configured to adjust an X-ray irradiation field formed by the X-rays generated from the X-ray generator in accordance with an opening area of a variable window and to maintain the X-ray irradiation field at a preset X-ray irradiation field size; a drive unit configured to operate the X-ray irradiation field limiting unit to adjust the X-ray irradiation field; a light irradiation field device mounted inside the main body and configured to form a light irradiation field aligned with the X-ray irradiation field through the variable window with respect to a subject disposed in front of the main body; a distance information acquiring device mounted on the main body and configured to measure and acquire a distance between a focus of the X-ray generator and the subject; and a controller configured to control an operation of the drive unit based on the distance received from the distance information acquiring device so that the X-ray irradiation field and the light irradiation field are adjusted by operating the X-ray irradiation field limiting unit.

The automatically controlled X-ray irradiation field limiting device further includes: an X-ray attenuation device mounted on the main body to attenuate the X-rays generated from the x-ray generator. The X-ray attenuation device includes: a turret table rotatably mounted inside the main body and provided with a plurality of holes formed along a circumferential direction of the turret table so that a center of each of the holes can be aligned with the central ray; a plurality of attenuation blocks 154 respectively mounted on the holes except for one of the holes so as to attenuate the X-rays; and a drive unit configured to rotate the turret table under the control of the controller. Since the X-rays are attenuated by the respective attenuation blocks having different attenuation coefficients, the cumulative radiation dose can be reduced depending on the subject, thereby minimizing damage to the human body.

The automatically controlled X-ray irradiation field limiting device according to the present invention controls the operation of the X-ray irradiation field limiting unit depending on the distance between the focus of the X-ray generator and the subject to automatically adjust and maintain the X-ray irradiation field at a preset X-ray irradiation field size. This makes it possible to minimize the exposure dose by reducing the amount of X-rays irradiated to unnecessary regions. In addition, a cumulative radiation dose is reduced by selecting one of attenuation blocks having different attenuation coefficients and selectively attenuating X-rays. This makes it possible to minimize the damage to a human body caused by the accumulation of radiation. It is therefore possible to enhance the safety and reliability of radiation examination and to improve the image quality.

### BRIEF DESCRIPTION OF THE DRAWINGS

FIG. 1 is a view schematically showing the configuration of an automatically controlled X-ray irradiation field limiting device according to the present invention.

FIG. 2 is a block diagram for explaining the control of the automatically controlled X-ray irradiation field limiting device according to the present invention.

FIGS. 3A and 3B are bottom views for explaining the operation of the automatically controlled X-ray irradiation field limiting device according to the present invention.

FIG. 4 is a perspective view showing an X-ray attenuator in the automatically controlled X-ray irradiation field limiting device according to the present invention.

FIG. 5 is a view for explaining the operation of the automatically controlled X-ray irradiation field limiting device according to the present invention.

FIG. 6 is a view for explaining a change of an X-ray irradiation field depending on an opening area of a variable window in the automatically controlled X-ray irradiation field limiting device according to the present invention.

FIG. 7 is a view for explaining the area dose depending on the distance between a focus and a subject in the automatically controlled X-ray irradiation field limiting device according to the present invention.

### DETAILED DESCRIPTION

Other objects, specific advantages and novel features of the present invention will become more apparent from the following detailed description of preferred embodiments made with reference to the accompanying drawings.

Hereinafter, preferred embodiments of an automatically controlled X-ray irradiation field limiting device according to the present invention will be described in detail with reference to the accompanying drawings.

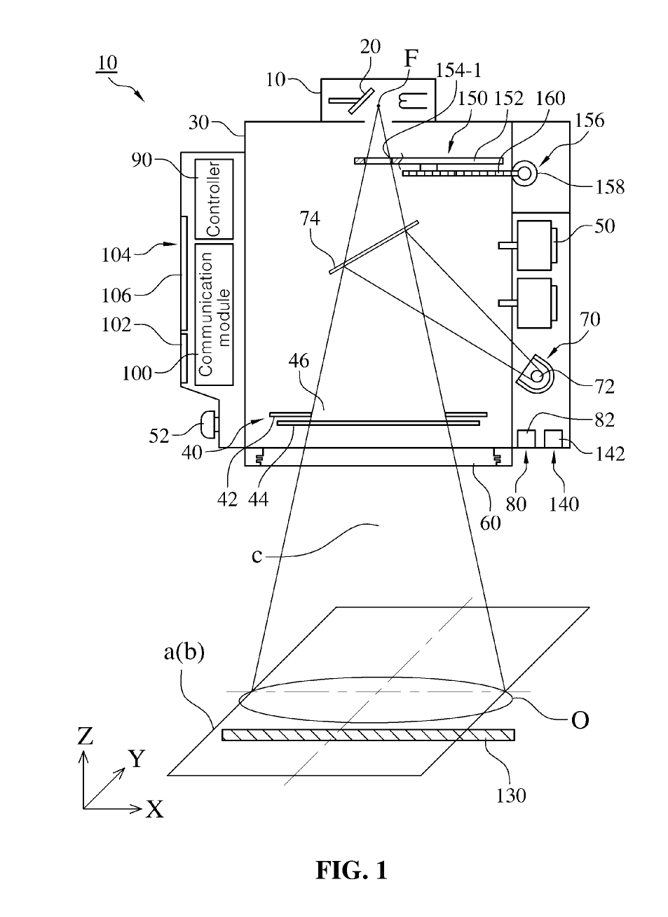

Referring first to FIG. 1, the automatically controlled X-ray irradiation field limiting device 10 according to the present invention includes a main body 30 disposed in front of an X-ray generator 20 for generating X-rays, and an X-ray irradiation field limiting unit 40 mounted inside the main body 30 so as to limit a X-ray irradiation field (a). The main body 30 or the housing may be made of a radiopacity material such as lead (Pb) or an alloy containing lead as a main component so as to shield X-rays against leakage. The inner surface and the outer surface of the main body 30 may be coated with a paint containing lead as a main component. In some embodiments, an auxiliary shielding mechanism such as a screen or a shield made of lead glass for shielding X-rays may be disposed around the X-ray generator 20 and the main body 30.

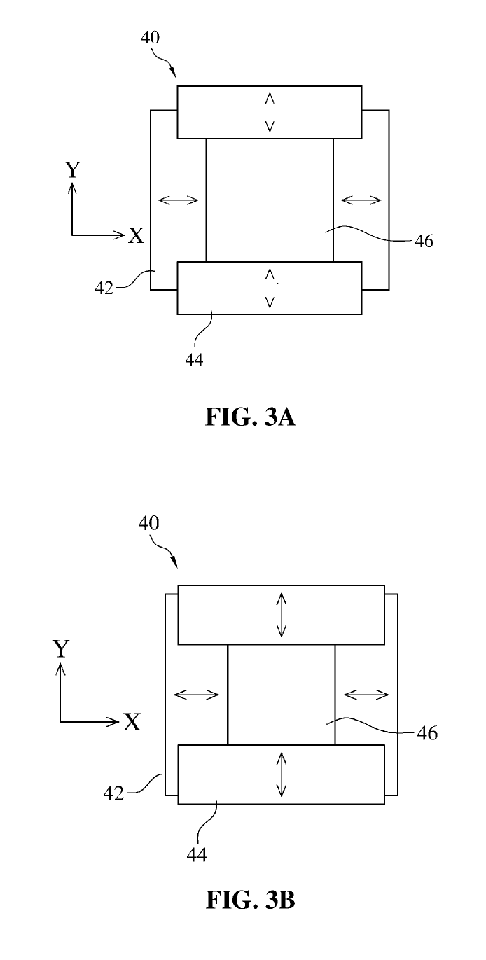

Referring to FIGS. 1 and 3, the X-ray irradiation field limiting unit 40 is disposed on the front side inside the main body 30 adjacent to a subject O, for example, a patient. The X-ray irradiation field limiting unit 40 or the collimator assembly includes a pair of first X-ray limiting blades 42 arranged parallel to each other along the Y direction, and a pair of second X-ray limiting blades 44 arranged parallel to each other along the X direction. The second X-ray limiting blades 44 are disposed in front of the first X-ray limiting blades 42 so as to overlap with the first X-ray limiting blades 42. Each of the first and second X-ray limiting blades 42 and 44 or the collimator blades may be made of lead or an alloy mainly composed of lead. As clearly shown in FIGS. 3A and 3B, the first and second X-ray limiting blades 42 and 44 are arranged on the upper and lower sides to form an X-ray-transmitting rectangular variable window 46 when seen in a plan view.

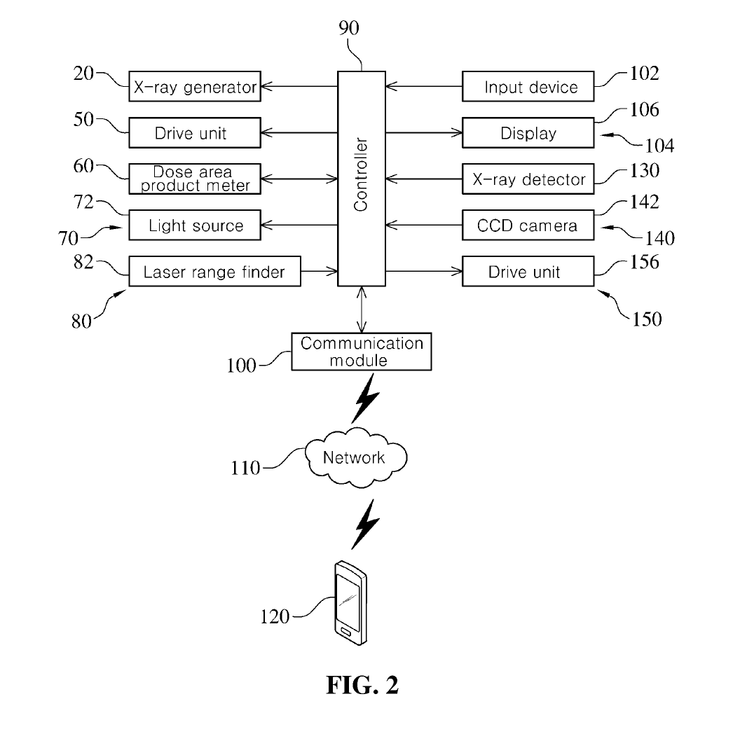

Referring to FIGS. 1 and 2, the automatically controlled X-ray irradiation field limiting device 10 according to the present invention includes a drive unit 40 for operating the X-ray irradiation field limiting unit 40 to adjust an X-ray irradiation field (a). The drive unit 50 moves the first and second X-ray limiting blades 44 and 46 toward or away from each other to adjust the opening area or opening rate of the variable window 46. The X-ray irradiation field (a) is determined according to the opening area of the variable window 46. The drive unit 50 may be formed of a screw-driven linear actuator, a rack and pinion actuator, a belt-driven linear actuator, a wire actuator, or the like.

Adjustment screw 52 pass through the main body 30 and are connected to the first and second X-ray limiting blades 42 and 44, respectively, so as to manually move the first and second X-ray limiting blades 42 and 44. By the rotation of the adjustment screws 52, the first and second X-ray limiting blades 42 and 44 are moved toward or away from each other just like an X-Y stage. The opening area of the variable window 46 is adjusted depending on the moving distance of each of the first and second X-ray limiting blades 42 and 44.

The automatically controlled X-ray irradiation field limiting device 10 according to the present invention includes a dose area product dosimeter or a dose area product meter 60 mounted inside the main body 30 in front of the X-ray irradiation field limiting unit 40 and configured to measure an X-ray dose. The dose area product meter 60 is an ion chamber device and is configured to measure the amount of X-rays passing through the variable window 46. The unit of an area dose with respect to the opening area of the variable window 46 may be denoted by Gy.m² (gray, Gy=J/kg).

The automatically controlled X-ray irradiation field limiting device 10 according to the present invention includes a light irradiation field device 70 for displaying an X-ray irradiation field (a), which is formed by the X-rays generated from the X-ray generator 20, in the form of a visual light irradiation field (b). The light irradiation field device 70 includes a light source 72 for projecting light and a reflective mirror 74. The light source 72 may be formed of a light emitting diode (LED), an organic light emitting diode (OLED), a halogen lamp, or the like, which is mounted on one side of the main body 30. The reflective mirror 74 is mounted inside the body 30 between the X-ray generator 20 and the X-ray irradiation field limiting unit 40 so as to reflect the light from the light source 72 toward the variable window 46. In some embodiments, the light irradiation field device 70 may be formed of a light source mounted on a collimator disposed in front of the X-ray generator 20.

The automatically controlled X-ray irradiation field limiting device 10 according to the present invention includes a distance information acquiring device 80 for measuring and acquiring the distance L from a focus F of the X-ray generator 20 to a subject O. The distance information acquiring device 80 may be formed of a laser range finder 82, which is mounted on the front end of the main body 30 adjacent to the subject O, a radio range finder, an ultrasonic distance measuring device, an infrared distance measuring device, or the like. Preferably, the distance information acquiring device 80 is formed of a laser range finder 82. The distance from the focus F of the X-ray generator 20 to the front end of the main body 30 and the distance from the focus F of the X-ray generator 20 to the X-ray irradiation field limiting unit 40 may be aware at the time of designing the automatically controlled X-ray irradiation field limiting device 10. The laser range finder 82 measures the distance from the front end of the main body 30 to the subject O. The distance L from the focus F to the subject O may be obtained by adding the distance from the focus F to the front end of the main body 30 and the distance from the front end of the main body 30 to the subject O, which is measured by the laser range finder 82.

The automatically controlled X-ray irradiation field limiting device 10 according to the present invention includes a controller 90 for controlling the X-ray generator 20, the drive unit 50, the dose area product meter 60, the light irradiation field device 70 and the distance information acquiring device 80. The controller 90 controls the operation of the drive unit 50 on the basis of the distance L between the focus F of the X-ray generator 20 and the subject O to adjust the distance between the first and second X-ray limiting blades 42 and 44. The opening area of the variable window 46 is adjusted in accordance with the distance between the first and second X-ray limiting blades 42 and 44 so that the X-ray irradiation field (a) and the light irradiation field (b) are adjusted. The controller 90 may be formed of a microcontroller or a microcontroller unit (MCU) mounted on one side of the main body 30.

The controller 90 is connected to a communication module 100, an input device 102 and an output device 104, respectively. The communication module 100 may be formed of a long term evolution module (LTE module), a Bluetooth module, a Wi-Fi module (wireless fidelity module), a zigbee module, or the like. The communication module 100 may be connected to an external terminal 120 through a wireless telecommunication network. The wireless telecommunication network may be formed of a long term evolution network (LTE network), a Bluetooth network, a Wi-Fi network (wireless fidelity network), a zigbee network, or the like.

The controller 90 may transmit a series of information required for the operation of the automatically controlled X-ray irradiation field limiting device 10 to the external terminal 120 via the communication module 100 and the network 110, or may receive and execute a series of information required for the operation of the automatically controlled X-ray irradiation field limiting device 10. An operator may control the controller 90 by the operation of the external terminal 120 to remotely control the operation of the automatically controlled X-ray irradiation field limiting device 10. The external terminal 120 may be formed of a mobile phone such as a smart phone, a cellular phone, a wireless application protocol phone (WAP phone) or the like, a handheld computer, a tablet computer, or the like, which is connected to the network 110. The external terminal 120 may also be formed of a computer system connected to the Internet via a network. In some embodiments, the external terminal 120 may be connected via a wired network.

The input device 102 may be composed of a control panel or a touch screen panel having a power switch, a button, a key pad and the like, which is capable of controlling the automatically controlled X-ray irradiation field limiting device 10 by the operation of an operator. The output device 104 may be formed of a display 106, a touch screen panel, an LED indicator, a speaker, a buzzer, and the like, which are capable of outputting a control mode of the automatically controlled X-ray irradiation field limiting device 10.

The automatically controlled X-ray irradiation field limiting device 10 according to the present invention includes an X-ray detector 130 disposed over a subject O to detect X-rays. The X-ray detector 130 may be composed of a film, an image intensifying tube, a flat panel detector, or the like.

The automatically controlled X-ray irradiation field limiting device 10 according to the present invention includes an image acquiring device 140 for acquiring an image of a subject O. The image acquisition device 140 is formed of a charge coupled device (CCD) camera 142 or a complementary metal oxide semiconductor (CMOS) camera mounted to the front end of the main body 30 so as to be connected to the controller 90. The CCD camera 142 captures an image of a light irradiation field (b) projected onto the subject O and inputs the data of the image to the controller 90.

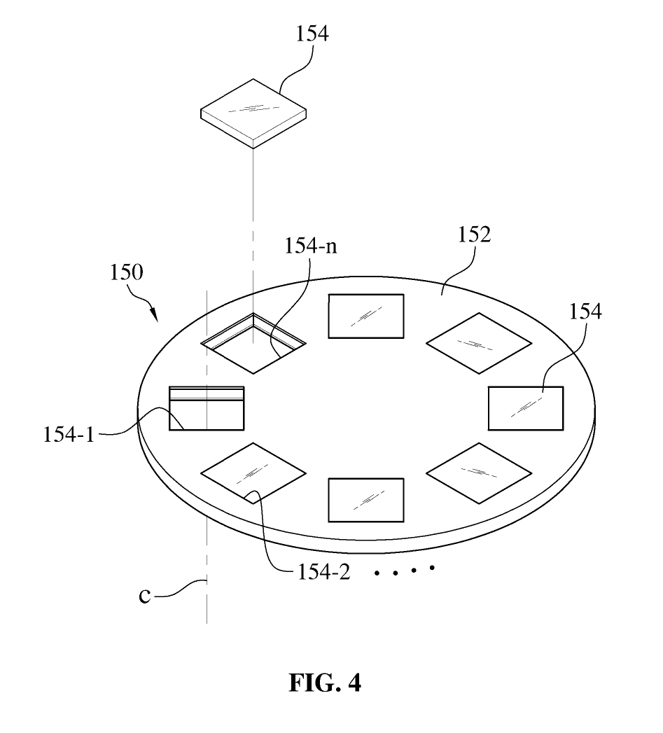

Referring to FIGS. 1 and 4, the automatically controlled X-ray irradiation field limiting device 10 according to the present invention further includes an X-ray attenuation device 150 for attenuating (reducing) the X-rays generated from the x-ray generator 20. A turret table 152 of the X-ray attenuation device 150 is rotatably disposed adjacent to the front side of the x-ray generator 20 and is arranged inside the main body 30 between the X-ray generator 20 and the X-ray irradiation field limiting unit 40. A plurality of holes 154-1, 154-2, . . . , 154-n, or windows are formed in the circumferential direction of the turret table 152. The center of each of the holes 154-1, 154-2, . . . , 154-n is aligned with the central ray (c).

The X-ray attenuation device 150 includes a plurality of attenuation blocks 154 having different attenuation coefficients and a drive unit 156. The respective attenuation blocks 154 are mounted to the holes 154-2, . . . , 154-n except for the hole 154-1. Each of the damping blocks 154 or the added filters may be formed of a plate made of aluminum (Al), molybdenum (Mo), niobium (Nb), zirconium (Zr), or an alloy thereof. The respective attenuation blocks 154 are configured to have different thicknesses so as to have different X-ray attenuation coefficients. That is, the ※ ray attenuation coefficient varies depending on the thickness of each of the attenuation blocks 154. Although the holes 154-1, 154-2, . . . , 154-n and the attenuation blocks 154 are shown as being square in FIG. 4, the holes 154-1, 154-2, . . . , 154-n and the attenuation blocks 154 may be formed in a circular shape. The drive unit 156 includes a servomotor 158 mounted on one side of the main body 30 and a gearing 160 for transmitting the driving power of the servo motor 158 to the turret table 152. The gearing 160 may be replaced by a belt drive.

Hereinafter, the operation of the automatically controlled X-ray irradiation field limiting device according to the present invention having such a configuration will be described.

Referring to FIGS. 1 and 2, the operator controls the operation of the automatically controlled X-ray irradiation field limiting device 10 by the operation of the input device 102 or the external terminal 120. The light from the light source 72 is reflected by the reflective mirror 74 and projected onto the subject O through the variable window 46 between the first and second X-ray limiting blades 42 and 44, whereby a light irradiation field (b) is formed on the surface of the subject O. Since the X-rays and the light reflected from the reflective mirror 74 pass through the variable window 46, the X-ray irradiation field (a) and the light irradiation field (b) coincide with each other. The operator can clearly confirm the exposure area of the subject O on which the X-rays are incident, based on the light irradiation field (b).

On the other hand, if the X-ray irradiation field (a) and the light irradiation field (b) are not coincident with each other, there may be a case where the X-ray irradiation field (a) has to be made wider than necessary. In this case, as the exposure dose increases, the generation of scattered rays increases and the image quality deteriorates. Therefore, the X-ray irradiation field (a) and the light irradiation field (b) should be aligned by adjusting the positions of the X-ray generator 20, the reflective mirror 74 and the like.

Subsequently, the CCD camera 142 captures an image of the light irradiation field (b) projected onto the subject O and transmits the image data to the controller 90. The controller 90 displays the image of the light irradiation field (b) through the display 106 or transmits the image data to the external terminal 120 through the communication module 100 and the network 110. The operator can accurately check the image of the light irradiation field (b) displayed through the external terminal 120 at a remote site to control the operation of the automatically controlled X-ray irradiation field limiting device 10.

Referring to FIGS. 1 and 4, when the hole 154-1 of the turret table 152 is aligned with the central ray c, the X-rays generated from the x-ray generator 20 pass through the hole 154-1. When the turret table 152 is rotated by driving the drive unit 156 and any one of the attenuation blocks 154 is aligned with the central ray c, the X-rays generated from the x-ray generator 20 are attenuated and irradiated according to the thickness of each of the attenuation blocks 154. The radiation exposure of the subject O can be reduced by the X-ray attenuation in each of the attenuation blocks 154. Therefore, by appropriately selecting the attenuation blocks 154 according to the cumulative radiation dose of the subject O and reducing the cumulative radiation dose, it is possible to minimize the body damage due to the accumulation of radiation. In addition, by selecting the attenuation blocks 154 so as to match the X-ray inspection area of the subject O and attenuating the X-rays, it is possible to enhance the contrast to improve the image quality.

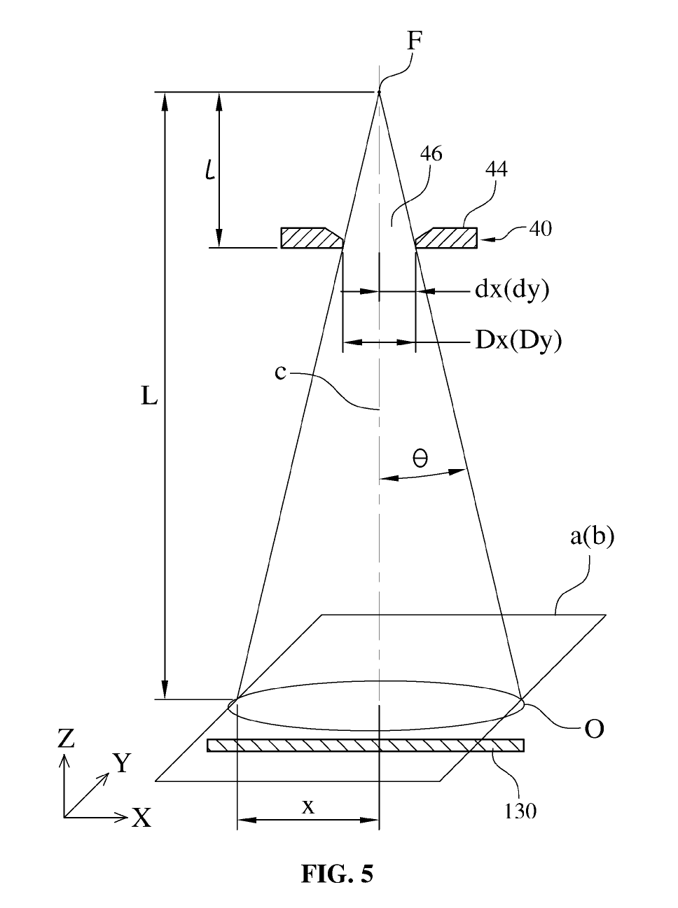

Referring to FIGS. 1 and 3A to 5, the laser range finder 82 measures the distance to the subject O and transmits the measured distance to the controller 90. The controller 90 processes the distance to the subject O inputted from the laser range finder 82 to obtain the distance L from the focus F of the X-ray generator 20 to the subject O. The controller 90 controls the operation of the drive unit 50 by setting the opening area or opening rate of the X-ray irradiation field limiting unit 40 based on the distance L from the focus F to the subject O. By the operation of the drive unit 50, the first and second X-ray limiting blades 42 and 44 are moved toward or away from each other to adjust the opening area of the variable window 46.

On the other hand, when the distance L is less than a reference set value, the controller 90 controls the operation of the drive unit 50 so that the opening area of the variable window 46 is increased. When the distance L exceeds the reference set value, the controller 90 controls the operation of the drive unit 50 so that the opening area of the variable window 46 is reduced. Thus, the X-ray exposure area can be optimally adjusted by adjusting the opening area of the variable window 46 in accordance with the distance L from the focus F to the subject O. In addition, by adjusting the opening area of the variable window 46 so as to match a preset X-ray irradiation field size, it is possible to keep the preset X-ray irradiation field size constant irrespective of the distance from the focus F to the subject O.

The X-ray irradiation field (a), i.e., the exposure area, according to the opening area of the variable window 46 can be obtained by the following equation.

tan(θx)=dx/l=x/L

x=(L·dx)l

tan(θy)=dy/l=y/L

y=(L·dy)/y/L [Equation 1]

In equation 1, l is the distance (mm) from the focus F to the subject O, L is the distance (mm) from the focus F to the exit of the variable window 46, x is the length (mm) in the x axis direction with respect to the subject O on the basis of the central ray c, y is the length (mm) in the Y axis direction with respect to the subject O on the basis of the central ray c, dx (Dx=2dx) is the length (mm) in the X axis direction with respect to the variable window 46 on the basis of the central ray c, dy (Dy=2dy) is the length (mm) in the Y axis direction with respect to the variable window 46 on the basis of the central ray c, θx is the angle formed by the X-ray in the X axis direction with respect to the central ray c, and θy is the angle formed by the X-ray in the Y axis direction with respect to the central ray c. Each of x and y is equal to the length (mm) in the X axis or Y axis direction with respect to the X-ray irradiation field (a) on the basis of the central ray c. In some embodiments, each of x and y may be detected by mounting an encoder or a variable resistor to the drive unit 50.

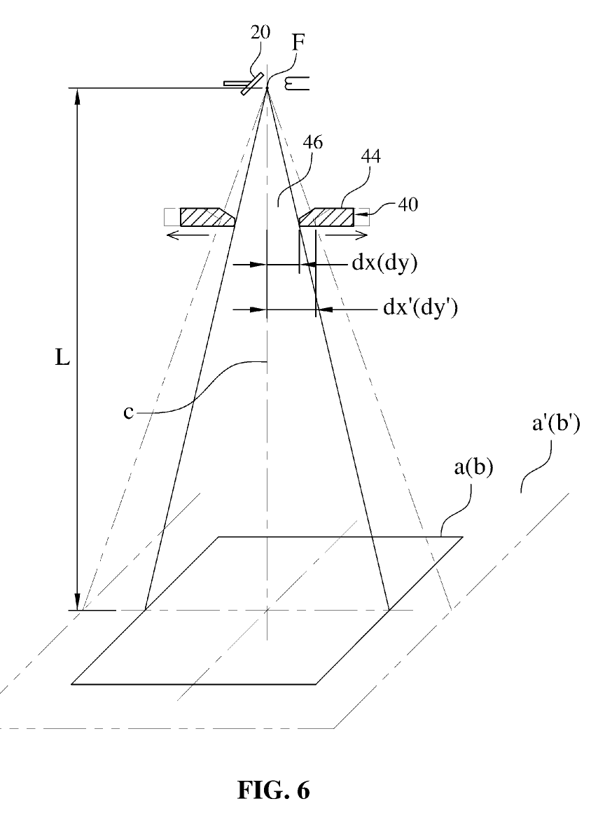

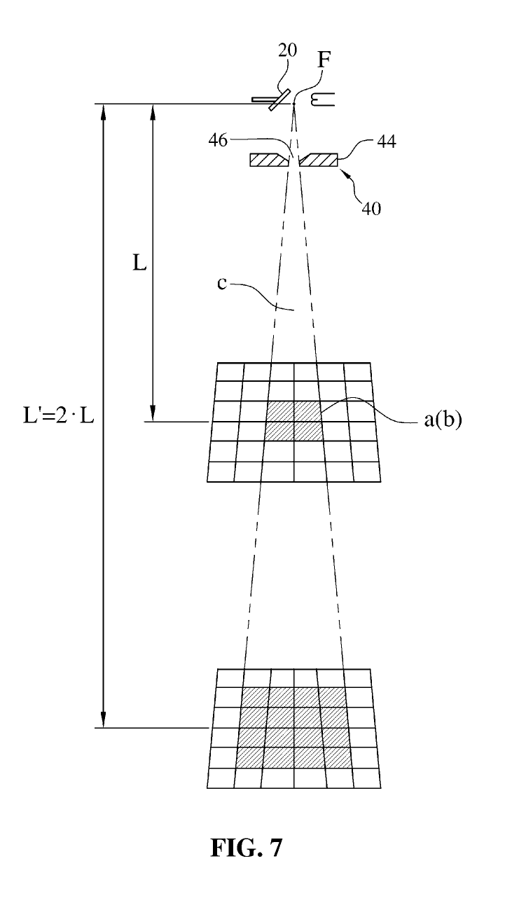

Referring to FIGS. 6 and 7, in the automatically controlled X-ray irradiation field limiting device 10 according to the present invention, when the distance L to the subject O is changed to the distance L′, dx, i.e., the length (mm) in the X axis direction with respect to the variable window 46 on the basis of the central ray c is changed to dx′, and dy, i.e., the length (mm) in the Y axis direction with respect to the variable window 46 on the basis of the central ray c is changed to dy′ under the control of the controller 90, whereby the X-ray irradiation field (a) is automatically adjusted. dx′ and dy′ can be obtained by the following equation.

dx′=(x·l)/L′

dy′=(y·l)/L [Equation 2]

In equation 2, L′ is the distance (mm) changed from the distance L from the focus F to the subject O. The distances L and L′ can be accurately obtained by the laser range finder 82. x and y can be set in advance.

Subsequently, the controller 90 controls the operation of the drive unit 50 based on the distances L and L′ measured by the laser range finder 82. The first and second X-ray limiting blades 42 and 44 are moved toward or away from each other or away from each other by the operation of the drive unit 50, whereby the distance between the first and second X-ray limiting blades 42 and 44 is adjusted and the opening area of the variable window 46 is automatically adjusted. By adjusting the opening area, it is possible to optimally control the X-ray irradiation field (a), i.e., the exposure area.

As shown in FIG. 7, the intensity of the X-rays irradiated through the X-ray irradiation field limiting unit 40 decreases in inverse proportion to the square of the distance. As the distance increases, the X-ray irradiation field (a), i.e., the X-ray irradiation area, increases. The area dose of the X-ray measured by the dose area product meter 60 is constant regardless of the distances L and L′ from the focus F to the subject O. That is, when the distance L is increased twice to the distance L′, the X-ray irradiation field (a) is also doubled. However, the area dose remains the same.

The standard dose Ig of the X-rays can be obtained by the following equation.

Ig=Sg/(4xy)

Ig=(l²·Sg)/(4L²·dx·dy) [Equation 3]

In equation 3, Sg is the area dose.

On the other hand, the controller 90 displays the standard dose on the display 106 or the external terminal 120 so that the operator can check the standard dose and control the automatically controlled X-ray irradiation field limiting device 10. By confirming the standard dose, the quality of the X-rays can be managed and the reliability of the examination for the subject O by the automatically controlled X-ray irradiation field limiting device 10 can be improved.

The embodiments described above are nothing more than preferred embodiments of the present invention and are not intended to limit the scope of the present invention. Those skilled in the art may make different changes, modifications or substitutions without departing from the spirit and scope of the present invention. It is to be understood that such changes, modifications or substitutions fall within the scope of the present invention.

What is claimed is:

1. An automatically controlled X-ray irradiation field limiting device, comprising:
a main body disposed in front of an X-ray generator for generating X-rays;
an X-ray irradiation field limiting unit mounted to the main body and configured to adjust an X-ray irradiation field formed by the X-rays generated from the X-ray generator in accordance with an opening area of a variable window and to maintain the X-ray irradiation field at a preset X-ray irradiation field size;
a drive unit configured to operate the X-ray irradiation field limiting unit to adjust the X-ray irradiation field;
a light irradiation field device mounted inside the main body and configured to form a light irradiation field aligned with the X-ray irradiation field through the variable window with respect to a subject disposed in front of the main body;
a distance information acquiring device mounted on the main body and configured to measure and acquire a distance between a focus of the X-ray generator and the subject; and
a controller configured to control an operation of the drive unit based on the distance received from the distance information acquiring device so that the X-ray irradiation field and the light irradiation field are adjusted by operating the X-ray irradiation field limiting unit.

2. The automatically controlled X-ray irradiation field limiting device of claim 1, wherein the controller controls the operation of the drive unit, and controls the operation of the drive unit to reduce the opening area of the variable window when the distance exceeds the reference set value.

3. The automatically controlled X-ray irradiation field limiting device of claim 1, wherein the light irradiation field device includes:
a light source mounted on one side of the main body so as to project light onto a central ray; and
a reflective mirror mounted inside the main body and configured to reflect the light from the light source so as to pass through the variable window.

4. The automatically controlled X-ray irradiation field limiting device of claim 1, further comprising:
a dose area product meter mounted on the main body and disposed in front of the X-ray irradiation field limiting unit to measure an area dose of the X-rays; and
an image acquiring device configured to acquire an image of the light irradiation field projected onto the subject and to transmit the image to the controller.

5. The automatically controlled X-ray irradiation field limiting device of claim 4, further comprising:
a communication device mounted on the main body and connected to the controller,
wherein the controller is configured to calculate a standard dose incident on the subject based on the distance from the distance information acquiring device and the area dose from the dose area product meter, and
the image of the light irradiation field and the standard dose are transmitted to an external terminal connected to the communication device via a network.

6. The automatically controlled X-ray irradiation field limiting device of claim 1, further comprising:
an X-ray attenuation device mounted on the main body to attenuate the X-rays generated from the x-ray generator.

7. The automatically controlled X-ray irradiation field limiting device of claim 6, wherein the X-ray attenuation device includes:
a turret table rotatably mounted inside the main body and provided with a plurality of holes formed along a circumferential direction of the turret table so that a center of each of the holes can be aligned with the central ray;
a plurality of attenuation blocks 154 respectively mounted on the holes except for one of the holes so as to attenuate the X-rays; and
a drive unit configured to rotate the turret table under the control of the controller.

8. The automatically controlled X-ray irradiation field limiting device of claim 7, wherein the attenuation blocks are formed of one of aluminum plates and aluminum alloy plates having different thicknesses so as to differ in X-ray attenuation coefficient from each other.

9. The automatically controlled X-ray irradiation field limiting device of claim 8, wherein the turret table is disposed between the X-ray generator and the X-ray irradiation field limiting unit so as to be adjacent to the X-ray generator.
